(19) United States
(12) Patent Application Publication
(10) Pub. No.: US 2024/0145028 A1
(43) Pub. Date: May 2, 2024

(54) Method For Data Augmentation Related To Target Protein

(71) Applicant: DEARGEN INC., Daejeon (KR)

(72) Inventors: Daeseok LEE, Seoul (KR); Bonggun SHIN, Gyeongsangbuk-do (KR)

(73) Assignee: DEARGEN INC., Daejeon (KR)

(21) Appl. No.: 17/973,918

(22) Filed: Oct. 26, 2022

(51) Int. Cl. G16B 15/30; G06N 3/08

(52) U.S. Cl. CPC G16B 15/30; G06N 3/08

(57) ABSTRACT

Disclosed is a computer program stored in a computer-readable storage medium. The method may include: obtaining a target protein included in training data and indicator information related to the target protein; identifying a homologous protein of the target protein; and augmenting the training data by matching the homologous protein to the indicator information related to the target protein.

### TECHNICAL FIELD

The present disclosure relates to a method of augmenting data, and more particularly, to a method of augmenting training data associated with a target protein.

### BACKGROUND ART

A drug target interaction (hereinafter, referred to as DTI) prediction problem is a problem in which a chemical affinity of a given drug molecule and a target protein is predicted in various schemes. For example, the DTI problem is a problem in which the chemical affinity measured in various schemes, such as IC₅₀, Kᵢ, K_{d}, or a modification value thereof between the given drug molecule and the target protein is computatively predicted. Meanwhile, in relation to the DTI problem, in a situation in which a structure of a target is known, a method such as docking is used, and in a situation in which the structure of the target is not known, this problem is defined and handled by various schemes a binary classification problem, a regressive problem, a bipartite graph inference problem, etc. In particular, in terms of the regressive problem, various machine learning and deep learning algorithms such as KronRLS, SimBoost, DeepDTA, and Dearzen's MT-DTI are used.

One of difficulties when approaching the DTI problem by deep learning is that the type of protein which may be used as the training data is not a lot. One evidence thereof is that the numbers of types of proteins which appear in KIBA and DAVIS datasets are not just 229 and 224, respectively. Another indirect evidence is that the numbers of types of GPCR and protein kinase of a category of the protein which becomes a main target of drug in the human body are just 790 and 500, respectively. Referring to FIG. 3, such a problem is important particularly in that a generalization capability of a deep learning model trained through limited data to a new target is significantly limitative.

Korean Patent Registration No. 10-2213670 (Feb. 2, 2021) discloses a method for drug-target interaction prediction.

### SUMMARY OF THE INVENTION

The present disclosure has been made in an effort to provide a method of augmenting data associated with a target protein, which is capable of extending (augmenting) data by utilizing a homologous protein in order to partially solve a problem which occurs due to the lack of the type of protein which may be used as training data.

Meanwhile, a technical problem to be solved by the present disclosure is not limited to the above-mentioned technical problem, and various technical problems can be included within the scope which is apparent to those skilled in the art from contents to be described below.

An exemplary embodiment of the present disclosure provides a method performed by a computing device. The method may include: obtaining a target protein included in training data and indicator information related to the target protein; identifying a homologous protein of the target protein; and augmenting the training data by matching the homologous protein to the indicator information related to the target protein.

Alternatively, indicator information related to the target protein may include affinity information between the target protein and a drug.

Alternatively, the method may further include filtering the augmented training data by considering the affinity information between the target protein and the drug and the affinity information and the homologous protein for the drug.

Alternatively, the filtering may include comparing affinity information between the target protein and the drug given from training data or predicted by a deep learning model and affinity information between the homologous protein and the drug predicted by the deep learning model, in a current batch of training data.

Alternatively, the filtering may include filtering data regarding the homologous protein having accuracy of a specific ranking or more among accuracy values which the homologous proteins in the batch have, and the accuracy values may be generated based on a comparison between the affinity information between the homologous protein and the drug predicted by the deep learning model and the affinity information between the target protein and the drug given from training data or predicted by the deep learning model.

Alternatively, the filtering may include performing filtering for the augmented training data from a middle of a training process of a deep learning model which is currently trained.

Alternatively, the identifying of the homologous protein of the target protein may further include performing multiple sequence alignment (MSA) for the target protein and multiple homologous proteins.

Alternatively, the performing of the MSA may include performing a search for the target protein and multiple homologous proteins satisfying a predetermined matching degree ratio.

Another exemplary embodiment of the present disclosure provides a computer program stored in a computer-readable storage medium. The computer program executes the following operations for augmenting data associated with a target protein when the computer program is executed by one or more processors and the operations may include: an operation of obtaining a target protein included in training data and indicator information related to the target protein; an operation of identifying a homologous protein of the target protein; and an operation of matching the homologous protein to the indicator information related to the target protein.

Still another exemplary embodiment of the present disclosure provides a computing device. The device may include: at least one processor; and a memory, and at least one processor may be configured to obtain a target protein included in training data and indicator information related to the target protein, identify a homologous protein of the target protein, and augment the training data by matching the homologous protein to the indicator information related to the target protein.

According to an exemplary embodiment of the present disclosure, it is possible to provide a method of augmenting data associated with a target protein, which is capable of extending (augmenting) data by utilizing a homologous protein in order to partially solve a problem which occurs due to the lack of training data.

Meanwhile, the effects of the present disclosure are not limited to the above-mentioned effects, and various effects can be included within the scope which is apparent to those skilled in the art from contents to be described below.

### BRIEF DESCRIPTION OF THE DRAWINGS

FIG. 1 is a block diagram of a computing device of augmenting data associated with a target protein according to an exemplary embodiment of the present disclosure.

FIG. 2 is a diagram schematically illustrating a method of augmenting data associated with a target protein according to an exemplary embodiment of the present disclosure.

FIG. 3 illustrates a first experimental result showing an effect of a method for data augmentation for drug-target affinity prediction according to an exemplary embodiment of the present disclosure.

FIG. 4 illustrates a second experimental result showing an effect of a method for data augmentation for drug-target affinity prediction according to an exemplary embodiment of the present disclosure.

FIG. 5 is a flowchart illustrating a method of augmenting data associated with a target protein according to an exemplary embodiment of the present disclosure.

FIG. 6 is a conceptual view illustrating a neural network according to an exemplary embodiment of the present disclosure.

FIG. 7 is a block diagram of a computing device according to an exemplary embodiment of the present disclosure.

### DETAILED DESCRIPTION

Hereinafter, various embodiments are described with reference to the drawings. In the present specification, various descriptions are presented for understanding the present disclosure. However, it is obvious that the embodiments may be carried out even without a particular description.

Terms, “component”, “module”, “system”, and the like used in the present specification indicate a computer-related entity, hardware, firmware, software, a combination of software and hardware, or execution of software. For example, a component may be a procedure executed in a processor, a processor, an object, an execution thread, a program, and/or a computer, but is not limited thereto. For example, both an application executed in a computing device and the computing device may be components. One or more components may reside within a processor and/or an execution thread. One component may be localized within one computer. One component may be distributed between two or more computers. Further, the components may be executed by various computer readable medium having various data structures stored therein. For example, components may communicate through local and/or remote processing according to a signal (for example, data transmitted to another system through a network, such as Internet, through data and/or a signal from one component interacting with another component in a local system and a distributed system) having one or more data packets.

A term “or” intends to mean comprehensive “or”, not exclusive “or”. That is, unless otherwise specified or when it is unclear in context, “X uses A or B” intends to mean one of the natural comprehensive substitutions. That is, when X uses A, X uses B, or X uses both A and B, “X uses A or B” may be applied to any one among the cases. Further, a term “and/or” used in the present specification shall be understood to designate and include all of the possible combinations of one or more items among the listed relevant items.

A term “include” and/or “including” shall be understood as meaning that a corresponding characteristic and/or a constituent element exists. Further, a term “include” and/or “including” means that a corresponding characteristic and/or a constituent element exists, but it shall be understood that the existence or an addition of one or more other characteristics, constituent elements, and/or a group thereof is not excluded. Further, unless otherwise specified or when it is unclear that a single form is indicated in context, the singular shall be construed to generally mean “one or more” in the present specification and the claims.

The term “at least one of A or B” should be interpreted to mean “a case including only A”, “a case including only B”, and “a case in which A and B are combined”.

Those skilled in the art need to recognize that various illustrative logical blocks, configurations, modules, circuits, means, logic, and algorithm steps described in connection with the embodiments disclosed herein may be additionally implemented as electronic hardware, computer software, or combinations of both sides. To clearly illustrate the interchangeability of hardware and software, various illustrative components, blocks, constitutions, means, logic, modules, circuits, and steps have been described above generally in terms of their functionalities. Whether the functionalities are implemented as the hardware or software depends on a specific application and design restrictions given to an entire system. Skilled artisans may implement the described functionalities in various ways for each particular application. However, such implementation decisions should not be interpreted as causing a departure from the scope of the present disclosure.

The description of the presented embodiments is provided so that those skilled in the art of the present disclosure use or implement the present disclosure. Various modifications to the embodiments will be apparent to those skilled in the art. Generic principles defined herein may be applied to other embodiments without departing from the scope of the present disclosure. Therefore, the present disclosure is not limited to the embodiments presented herein. The present disclosure should be analyzed within the widest range which is coherent with the principles and new features presented herein.

In the present disclosure, a network function, an artificial neural network, and a neural network may be interchangeably used.

In this disclosure, an “indicator” related to a target protein is a concept illustrating various information related to characteristics of the target protein. The indicator related to the target protein may indicate various information including information on characteristics of the target protein itself, information on characteristics between the target protein and another material, information on a material closely related to the target protein, etc., and may be expressed by various schemes including an index, a scale, a measured value, etc. As an example, the indicator related to the target protein may include an “affinity” between the target protein and a compound.

In the present disclosure, the “affinity” is a concept representing various relationships (e.g., a binding possibility, relevance, correlation, reactivity, interaction, etc.) between a biological target and the compound may be determined based on various indexes, scales, or measure values. For example, the affinity may be determined based on various indexes, scales, or measure values including binding affinity, IC50 (half maximal inhibitory concentration), EC50 (half maximal effective concentration), AC50 (half activity concentration), etc.

In the present disclosure, the binding affinity which may be included in the affinity may mean a binding strength between multiple molecules which are reversibly bound, which is a kind of a reaction degree scale between the biological target and the compound. In general, it is known that there is a high probability that as a compound having a higher binding affinity will be specifically and selectively bound to the biological target. Further, the binding affinity may be a strength of a binding action of a protein or DNA and drug or inhibitor. Further, the binding affinity may be measured, for example, based on an equilibrium dissociation constant (K_{D}). In this case, it is expressed that as the K_{D} value is smaller, the binding affinity of the drug or inhibitor for the biological target is higher, and on the contrary, it may be expressed that as the K_{D} value is larger, the binding affinity of the drug or inhibitor for the biological target is lower. Further, the binding affinity may be influenced by an interaction between molecules non-covalently bound between two molecules, such as hydrogen binding, static electricity interaction, and hdyrophobicity. Further, the binding affinity may be used by measuring a physical sample through experimental and measurement devices, but used by using a database storing a measured value. As a scheme of measuring the binding affinity, various schemes may be used in addition to a scheme based on the equilibrium dissociation constant (K_{D}), and the present disclosure includes various schemes of measuring the binding affinity.

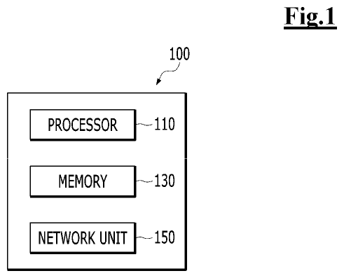

Meanwhile, as a more specific example, the “affinity” in the present disclosure may indicate a binding force, a catalyst speed, substrate specificity, chemical selectivity, receptor effect action, or receptor antagonist which acts between the drug and the target material. Further, here, the target material may be a protein such as a receptor, and the drug may serve as a ligand which may form a stable complex between the drug and the target material by interacting with a binding portion of the target material, and the complex may include coenzymes or auxiliary factors such as a metal ion in addition the drug-target material. Further, the ligand may be a small molecule which may be non-covalently bound with a target biomolecule for a pharmacological purpose, and may also be a biomolecule such as a nucleotide polymer, peptide, antibody, etc. Hereinafter, a method of augmenting data associated with a target protein performed by a computing device according to the present disclosure will be described through FIGS. 1 to 7.

FIG. 1 is a block diagram of a computing device of augmenting data associated with a target protein according to an exemplary embodiment of the present disclosure.

A configuration of the computing device 100 illustrated in FIG. 1 is only an example shown through simplification. In an exemplary embodiment of the present disclosure, the computing device 100 may include other components for performing a computing environment of the computing device 100 and only some of the disclosed components may constitute the computing device 100.

The computing device 100 may include a processor 110, a memory 130, and a network unit 150.

The processor 110 may be constituted by one or more cores and may include processors for data analysis and deep learning, which include a central processing unit (CPU), a general purpose graphics processing unit (GPGPU), a tensor processing unit (TPU), and the like of the computing device. The processor 110 may read a computer program stored in the memory 130 to perform data processing for machine learning according to an exemplary embodiment of the present disclosure. According to an exemplary embodiment of the present disclosure, the processor 110 may perform a calculation for learning the neural network. The processor 110 may perform calculations for learning the neural network, which include processing of input data for learning in deep learning (DL), extracting a feature in the input data, calculating an error, updating a weight of the neural network using backpropagation, and the like. At least one of the CPU, GPGPU, and TPU of the processor 110 may process learning of a network function. For example, both the CPU and the GPGPU may process the learning of the network function and data classification using the network function. Further, in an exemplary embodiment of the present disclosure, processors of a plurality of computing devices may be used together to process the learning of the network function and the data classification using the network function. Further, the computer program executed in the computing device according to an exemplary embodiment of the present disclosure may be a CPU, GPGPU, or TPU executable program.

According to an exemplary embodiment of the present disclosure, a method of augmenting data associated with a target protein performed by the processor 110 may be appreciated as a kind of weakly supervised learning. The weakly supervised learning is to utilize a label which is not accurate in any sense when training a machine learning model. As an example, there is a case of using a label obtained through crowd sourcing. According to an exemplary embodiment of the present disclosure, since the processor 110 augments training data by making affinity information between “target protein” and a given drug correspond to “homologous protein” of the target protein, the augmentation of the training data may be appreciated as a kind of weakly supervised learning (in terms of matching affinity information of another protein which is not relatively accurate other than accurate affinity information in view of the homologous protein).

The processor 110 may obtain the target protein included in the training data and indicator information related to the target protein, and identify the homologous protein of the target protein. Thereafter, the processor 110 may augment the training data by matching the homologous protein to the indicator information related to the target protein. In this case, the indicator information related to the target protein may include the affinity information between the drug and the target protein. For reference, the processor 110 may augment the training data by utilizing even any indicator which may be assumed that a corresponding value will also be similar when a protein structure is similar.

Meanwhile, the processor 110 uses the homologous protein in order to partially solve a problem in that the types of proteins which may be used as the training data are not a lot. As an example, if target protein A in the training data has a high affinity with drug X, there is a high possibility that protein B having a similar sequence pattern to target protein A has a high affinity with drug X. The reason is that there is a high possibility that protein B will also have a stereoscopic structure coupled with drug X which target protein A has. Therefore, the processor 110 may extend (augment) data including the homologous protein in a training data set used when training a pre-trained deep learning model (e.g., a DTI deep learning model). Specifically, the processor 110 may train deep learning model by assuming that an affinity value of (B, X) is a if the affinity value of (A, X) is a.

Additionally, in relation to extending the data by utilizing the homologous protein, there may also be a problem in that a portion where target protein A is coupled to drug X may be omitted or deformed in homologous protein B. In this case, an indicator (e.g., affinity) of homologous protein B and drug X will not be estimated through an indicator (e.g., affinity) of target protein A and drug X. Therefore, the training data set extended (augmented) by the above-described method is not used as it is, but it is necessary to appropriately filter the training data set. The processor according to an exemplary embodiment of the present disclosure may filter some of data augmented by utilizing the homologous protein in order to solve the problem, and through such a filtering operation, data (in which relative high accuracy may not be guaranteed) is prevented from being included in the training data to additionally improve performance of data augmentation.

According to an exemplary embodiment of the present disclosure, the memory 130 may store any type of information generated or determined by the processor 110 or any type of information received by the network unit 150.

According to an exemplary embodiment of the present disclosure, the memory 130 may include at least one type of storage medium of a flash memory type storage medium, a hard disk type storage medium, a multimedia card micro type storage medium, a card type memory (for example, an SD or XD memory, or the like), a random access memory (RAM), a static random access memory (SRAM), a read-only memory (ROM), an electrically erasable programmable read-only memory (EEPROM), a programmable read-only memory (PROM), a magnetic memory, a magnetic disk, and an optical disk. The computing device 100 may operate in connection with a web storage performing a storing function of the memory 130 on the Internet. The description of the memory is just an example and the present disclosure is not limited thereto.

The network unit 150 according to an exemplary embodiment of the present disclosure may use various wired communication systems such as public switched telephone network (PSTN), x digital subscriber line (xDSL), rate adaptive DSL (RADSL), multi rate DSL (MDSL), very high-speed DSL (VDSL), universal asymmetric DSL (UADSL), high bit rate DSL (HDSL), and local area network (LAN).

The network unit 150 presented in this specification may use various wireless communication systems such as code division multi access (CDMA), time division multi access (TDMA), frequency division multi access (FDMA), orthogonal frequency division multi access (OFDMA), single carrier-FDMA (SC-FDMA), and other systems.

In the present disclosure, the network unit 150 may be configured regardless of communication modes such as wired and wireless modes and constituted by various communication networks including a local area network (LAN), a personal area network (PAN), a wide area network (WAN), and the like. Further, the network may be known World Wide Web (WWW) and may adopt a wireless transmission technology used for short-distance communication, such as infrared data association (IrDA) or Bluetooth.

The techniques described in this specification may also be used in other networks in addition to the aforementioned networks.

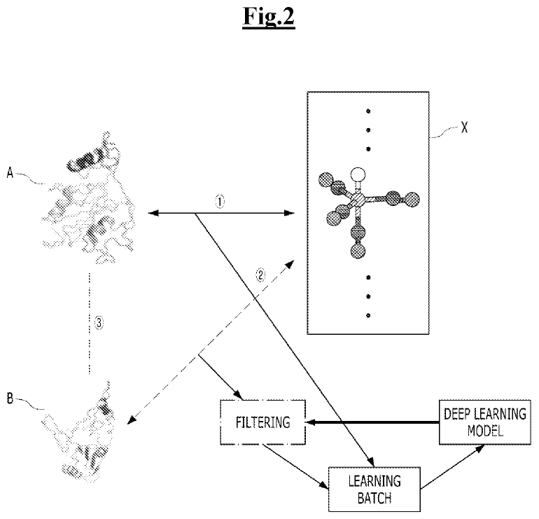

FIG. 2 is a diagram schematically illustrating a method of augmenting data associated with a target protein according to an exemplary embodiment of the present disclosure.

According to an exemplary embodiment of the present disclosure, the processor 110 may obtain a target protein and indicator information related to the target protein included in training data. In this case, the indicator information related to the target protein may include various indicators that may be assumed that a corresponding value will also be similar when a target protein structure and a protein structure are similar. As an example, the indicator information related to the target protein may include the affinity information between the drug and the target protein. Further, the processor 110 may identify the target protein and the homologous protein, and augment the training data by matching the homologous protein to the indicator information related to the target protein. That is, the processor 110 may augmenting the training data by matching the homologous protein to the indicator information related to the target protein.

As an example, referring to FIG. 2, the target protein A and the drug X has a known interactions (\textcircled{1}) relationship. For reference, since the target protein A and the drug X has the known interactions (\textcircled{1}) relationship, the target protein A and the indicator (e.g., affinity information a between the drug X and the target protein) related to the target protein are existing known information. Further, the target protein A and a homologous protein B as proteins having a similar sequence pattern have a homology (\textcircled{3}) relationship with each other. For reference, “similar” in the present disclosure may mean having a sequence matching degree of a predetermined ratio or more. In other words, the protein A and the homologous protein B as proteins in which the sequence pattern has the sequence matching degree of the predetermined ratio or more have the homology relationship with each other. Further, since the target protein A and the homologous protein B are proteins in which the sequence patterns are similar, the affinity values for the homologous protein B and the drug X may be assumed as affinity information a, so the homologous protein B and the drug X have a potential interaction (\textcircled{2}) relationship.

According to an exemplary embodiment of the present disclosure, the processor 110 may obtain affinity information for the target protein A and the drug X of the target protein included in training data. The affinity information may include information on binding force or force (strength) which act between the target protein A and the drug X, and include various types of information in addition to the information. For example, the affinity information may include various types of information including K_{D} (equilibrium dissociation constant), Kᵢ, IC50 (half maximal inhibitory concentration), EC50 (half maximal effective concentration), AC50 (half activity concentration), etc.

According to an exemplary embodiment of the present disclosure, the processor 110 may identify the homologous protein B of the target protein A. Further, the processor 110 may perform multiple sequence alignment (MSA) for the target protein and multiple homologous proteins. For example, the processor 110 may perform multiple sequence alignment (MSA) by searching a protein which is homologous with the target protein A in a database. The MSA is used in a method using an evolutionary correlation, a template-based method, etc., among approach methods of a protein structure prediction problem. Further, a structure prediction method using the homologous protein is also applied to a drug-target interaction prediction problem. The processor 110 may use the MSA for identifying the protein which is homologous with the target protein A among multiple proteins. As an example, the processor 110 may perform homologous structure search and multiple sequence alignment (MSA) by using an HHBlits algorithm based on a hidden Markov model, but the present disclosure is not limited, and an algorithm which is pre-developed or developed afterwards may be applied. Further, the processor 110 may search the target protein and multiple homologous proteins satisfying a predetermined matching degree ratio. As an example, the processor 110 may use homologous protein search by setting a minimum corresponding ratio to 70% of the HHBlits algorithm. The processor 110 may determine a predetermined ratio by finding a balance of variety and accuracy of data extension. The predetermined ratio may be a numerical value determined through prediction performance of a pre-trained deep learning model. However, the predetermined ratio is just an exemplary embodiment, and is not limited thereto.

According to an exemplary embodiment of the present disclosure, the processor 110 may augment (extend) the training data by matching the homologous protein to the indicator information related to the target protein. The processor 110 may augment (extend) the training data by using the homologous protein in order to partially solve a problem in that the types of proteins which may be used as the training data are not a lot. As an example, if target protein A in the training data has a high affinity with drug X, there is a high possibility that protein B having a similar sequence pattern to target protein A has a high affinity with drug X. The reason is that there is a high possibility that protein B will also have a stereoscopic structure coupled with drug X which target protein A has. Therefore, the processor 110 may extend (augment) data by including the homologous protein in a training data set used when training a pre-trained deep learning model (e.g., a DTI deep learning model). As an example, the processor 110 may train deep learning model by assuming that the affinity value of (B, X) is also a if the affinity value of (A, X) is a.

According to an exemplary embodiment of the present disclosure, the processor 110 may filter the augmented training data by considering the affinity information between the drug and the target protein and the affinity information between the drug and the homologous protein. The processor 110 may perform filtering in order to solve the problem of the augmented training data. A problem may occur in which the portion where the target protein A is coupled to the drug X may be omitted or deformed in the homologous protein B. In this case, the affinity between the homologous protein B and the drug X will not be estimated through the affinity between the target protein A and the drug X. Therefore, the training data set extended (augmented) by the above-described method is not used as it is, but it is necessary to appropriately filter the training data set. Meanwhile, the processor 110 may use (B, X, a) for training only when the deep learning model which is being trained predicts the affinity between the homologous protein B and the drug X comparatively close to the affinity a between the given target protein A and the drug X in data set.

According to an exemplary embodiment, the processor 110 may compare “affinity information between the target protein and the drug predicted by the deep learning model” and “affinity information between the homologous protein and the drug predicted by the deep learning model” in a current batch of training data. Alternatively, when the affinity information between the target protein and the drug is already included in the training data, the processor 110 may also compare the “affinity information between the target protein and the drug (included in the training data)” and the “affinity information between the homologous protein and the drug predicted by the deep learning model”. Further, the processor 110 may filter data regarding the homologous protein having accuracy of a specific ranking or more (e.g., an intermediate value or more) among accuracy values which the homologous proteins in the batch have. The processor 110 filters the data regarding the homologous protein having the accuracy of the specific ranking or more to enhance accuracy of learning. Here, the accuracy values may be generated by comparing the affinity information between the homologous protein and the drug predicted by the deep learning model, and the affinity information between the target protein and the drug predicted by the deep learning model or already included in the training data.

According to an exemplary embodiment of the present disclosure, the processor 110 may perform filtering for the augmented training data, but perform filtering from a middle of a training process of the deep learning model which is currently trained. In other words, the processor 110 may not perform the filtering operation from the start of the training for the deep learning model, but perform the filtering operation from any time point (i.e., any time point after the training is conducted at any degree) after the training starts, in relation to the augmented training data. The reason is that since the accuracy of the prediction of the deep learning model may not be sufficiently guaranteed in initial training, it is preferable not to perform the filtering operation by utilizing the deep learning model up to a predetermined time point from the start of the training, and it is preferable to perform the filtering operation by utilizing the deep learning model after the predetermined time point (i.e., a time point when the training is conducted at any degree and the accuracy of the prediction of the deep learning model may be guaranteed at any degree). As an example, the processor 110 may not perform the filtering operation for the augmented training data up to a predetermined epoch (e.g., 850 epochs) from the start of the training in the training process of the deep learning model which is currently trained, and perform the filtering operation after the predetermined epoch. Meanwhile, since data capable of reducing the accuracy of the prediction among the augmented training data may be removed through such a filtering operation, final performance of the deep learning model may be further improved.

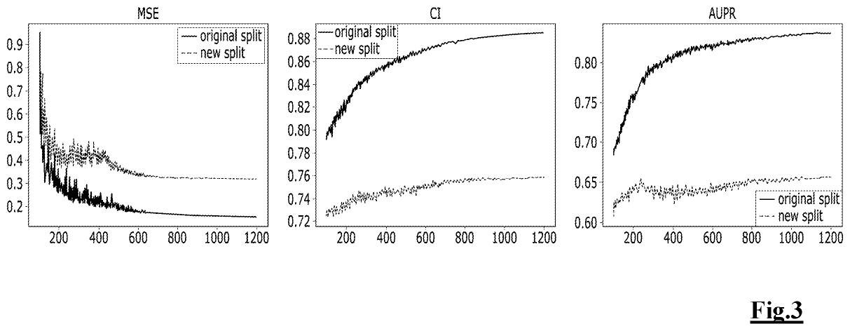

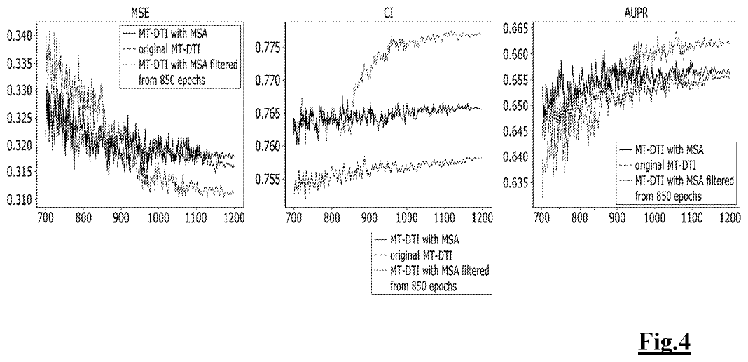

FIG. 3 illustrates a first experimental result showing an effect of a method for data augmentation for drug-target affinity prediction according to an exemplary embodiment of the present disclosure and FIG. 4 illustrates a second experimental result showing an effect of a method for data augmentation for drug-target affinity prediction according to an exemplary embodiment of the present disclosure. FIGS. 3 and 4 illustrate a performance evaluation result performed for all performance measurement items (e.g., MSE, CI, AUPR) based on different data sets. As a performance evaluation indicator, Mean square error (MSE), consistency index (CI), and area under precision-recall (AUPR) are used.

As an example, referring to FIG. 3, a first curve (original split) is a learning curve of a molecule transformer drug target interaction (MT-DIT) model in an existing KIBA data set. A second curve (new split) is an MT-DTI learning curve in a data set (hereinafter, referred to as “new data set”) obtained by dividing the KIBA data set so that only a new protein appears upon test. It may be confirmed that a generalization capability to a new target of the deep learning model learned through data limited through the learning curve of the original split is significantly limitative.

As an example, referring to FIG. 4, a third curve (original MT-DTI) is an MT-DTI learning curve in a new data set. The third curve (original MT-DTI) is the same as the second curve (new split) of FIG. 3. A fourth curve (MT-DTI with MSA) is a learning curve when multiple sequence alignment (MSA) information is additionally used in the new data set. In other words, the fourth curve (MT-DTI with MSA) is a learning curve when the training data is augmented by using the homologous protein in the method for data augmentation for drug-target affinity prediction (before using filtering) according to an exemplary embodiment of the present disclosure. A fifth curve (MT-DTI with MSA filtered from 850 epochs) is a learning curve when the multiple sequence alignment (MSA) information is additionally used in the new data set, and the filtering is performed. In other words, the fifth curve (MT-DTI with MSA filtered from 850 epochs) is a learning curve in which the training data is augmented by using the homologous protein and the filtering operation is additionally applied according to an exemplary embodiment of the present disclosure. For reference, the filtering is applied from 850 epochs. Referring to the learning curve of the fifth curve (MT-DTI with MSA filtered from 850 epochs), it may be confirmed that all performance measurement items (e.g., MSE, CI, and AUPR) shows excellent performance. The learning curve of FIG. 4 as a mean of five results learned by using four different unions of five learning folds means that the performance is excellent as the MSE is smaller and the CI and the AUPR are larger. That is, as illustrated in FIG. 4, “the augmented training data is utilized based on the homologous protein” and additionally, “the filtering is performed by considering the affinity information between the target protein and the drug and the affinity information between the homologous protein and the drug” to enhance the performance of the model.

According to an exemplary embodiment of the present disclosure, by using the existing KIBA data set (e.g., binding affinity related data set), the training is conducted by three following methods by using the same deep learning model to compare the performance of the model learned in the test set by using MSE, CI, AUPR, etc. Three methods may include \textcircled{1} a training method in the existing KIBA data set, \textcircled{2} a method of applying element 1 (e.g., data set extension using MSA), and \textcircled{3} a training method of applying element 1 (e.g., data set extension using MSA) and element 2 (e.g., filtering of the extended data set). In this case, a hold-out cross validation method may be used for validation, but the present disclosure is not limited thereto. For example, when the hold-out cross validation is used, all data sets may be divided into one test set and k train sets. More specifically, when k=3, (1) “training by train set 2 and train set 3=>evaluation by test set”, (2) “training by train set 1 and train set 3=>evaluation by test set”, and (3) “training by train set 1 and train set 2=>evaluation by test set” may be performed. In this case, an artificial intelligent model (deep learning model) or a training method may be evaluated by using a mean of scores output from respective evaluations ((1), (2), . . . , (k)) (for reference, in the case of FIG. 4, a scheme in which k=5 is used). Meanwhile, when the performance of (2) is better than the performance of (1)” is shown as a cross-validation result, it may be determined that element 1 is effective. Further, when “the performance of (3) is better than the performance of (2)” is shown, it may be determined that element 2 is effective.

Hereinafter, an operational flow of the present disclosure will be briefly described based on the contents described above in detail.

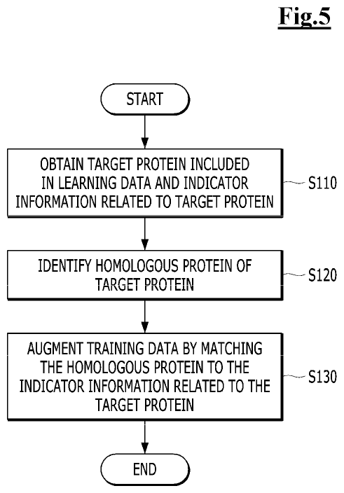

FIG. 5 is a flowchart illustrating a method of augmenting data associated with a target protein according to an exemplary embodiment of the present disclosure.

A method of augmenting data associated with the target protein illustrated in FIG. 5 may be performed by the computing device 100 described above. Therefore, in spite of the contents omitted below, the contents described for the computing device 100 may also be equally applied to the description of the method of augmenting data associated with the target protein.

According to an exemplary embodiment of the present disclosure, the computing device 100 may obtain a target protein included in training data and indicator information related to the target protein (S110). As an example, an indicator related to the target protein may include various information that may be assumed that a corresponding value will also be similar when a target protein structure and a protein structure are similar. Further, indicator information related to the target protein may include affinity information between the target protein and a drug.

The computing device 100 according to an exemplary embodiment of the present disclosure may identify a homologous protein of the target protein (S120). Here, the computing device 100 may perform multiple sequence alignment (MSA) for the target protein and multiple homologous proteins. Further, the MSA may be performed by search the target protein and multiple homologous proteins satisfying a predetermined matching degree ratio.

The computing device 100 according to an exemplary embodiment of the present disclosure may augment the training data by matching the homologous protein to the indicator information related to the target protein (S130). That is, the computing device 100 may augment data of a corresponding indicator by matching the homologous protein to the indicator information related to the target protein.

Meanwhile, the computing device 100 may filter the augmented training data by considering the affinity information between the drug and the target protein and the affinity information between the drug and the homologous protein. In this case, the computing device 100 may calculate accuracy information by comparing affinity information between the target protein and the drug given from training data or predicted by the deep learning model, and affinity information between the homologous protein and the drug predicted by the deep learning model, in a current batch of training data. And computing device 100 may use the calculated accuracy information for filtering. Further, the computing device 100 may filter data regarding the homologous protein having accuracy of a specific ranking or more among accuracy values which the homologous proteins in the batch have. Further, the computing device 100 may filtering for training data augmented from in a middle of a training process of the deep learning model which is currently learned.

In the above description, steps S110 to S130 may be further divided into additional steps or combined into fewer steps, according to an implementation example of the present disclosure. In addition, some steps may be omitted as necessary, and the order between the steps may be changed.

Meanwhile, in the disclosure described above, a weakly supervised learning scheme is used based on data augmentation of making the given affinity information between the target protein and the drug correspond to the homologous protein for the target protein, but the filtering operation is not performed from the first, but the filtering operation for the augmented data is performed from a step after the middle (e.g., after 800 epochs) of the training to improve the performance of a neural network model for drug-target affinity prediction.

In the present disclosure described above, the training data may be augmented and the performance of the neural network model may be improved without a need of comparing or mutually projecting the structures of the homologous proteins. Therefore, in the present disclosure, the performance of the neural network model may be improved while preventing excessive consumption of training resources which may be caused in the process of analyzing the structures of the homologous proteins in the augmentation process of the training data, mutually projecting the structures, and analyzing a phantom structure.

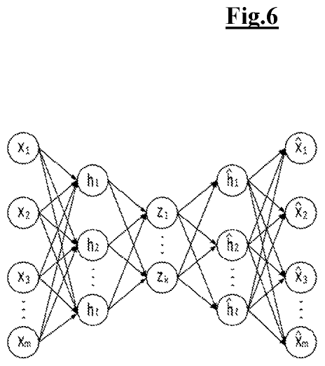

FIG. 6 is a schematic diagram illustrating a network function according to the embodiment of the present disclosure.

Throughout the present specification, the meanings of a calculation model, a nerve network, the network function, and the neural network may be interchangeably used. The neural network may be formed of a set of interconnected calculation units which are generally referred to as “nodes”. The “nodes” may also be called “neurons”. The neural network consists of one or more nodes. The nodes (or neurons) configuring the neural network may be interconnected by one or more links.

In the neural network, one or more nodes connected through the links may relatively form a relationship of an input node and an output node. The concept of the input node is relative to the concept of the output node, and a predetermined node having an output node relationship with respect to one node may have an input node relationship in a relationship with another node, and a reverse relationship is also available. As described above, the relationship between the input node and the output node may be generated based on the link. One or more output nodes may be connected to one input node through a link, and a reverse case may also be valid.

In the relationship between an input node and an output node connected through one link, a value of the output node data may be determined based on data input to the input node. Herein, a link connecting the input node and the output node may have a weight. The weight is variable, and in order for the neural network to perform a desired function, the weight may be varied by a user or an algorithm. For example, when one or more input nodes are connected to one output node by links, respectively, a value of the output node may be determined based on values input to the input nodes connected to the output node and weights set in the link corresponding to each of the input nodes.

As described above, in the neural network, one or more nodes are connected with each other through one or more links to form a relationship of an input node and an output node in the neural network. A characteristic of the neural network may be determined according to the number of nodes and links in the neural network, a correlation between the nodes and the links, and a value of the weight assigned to each of the links. For example, when there are two neural networks in which the numbers of nodes and links are the same and the weight values between the links are different, the two neural networks may be recognized to be different from each other.

The neural network may consist of a set of one or more nodes. A subset of the nodes configuring the neural network may form a layer. Some of the nodes configuring the neural network may form one layer on the basis of distances from an initial input node. For example, a set of nodes having a distance of n from an initial input node may form n layers. The distance from the initial input node may be defined by the minimum number of links, which need to be passed to reach a corresponding node from the initial input node. However, the definition of the layer is arbitrary for the description, and a degree of the layer in the neural network may be defined by a different method from the foregoing method. For example, the layers of the nodes may be defined by a distance from a final output node.

The initial input node may mean one or more nodes to which data is directly input without passing through a link in a relationship with other nodes among the nodes in the neural network. Otherwise, the initial input node may mean nodes which do not have other input nodes connected through the links in a relationship between the nodes based on the link in the neural network. Similarly, the final output node may mean one or more nodes that do not have an output node in a relationship with other nodes among the nodes in the neural network. Further, the hidden node may mean nodes configuring the neural network, not the initial input node and the final output node.

In the neural network according to the embodiment of the present disclosure, the number of nodes of the input layer may be the same as the number of nodes of the output layer, and the neural network may be in the form that the number of nodes decreases and then increases again from the input layer to the hidden layer. Further, in the neural network according to another embodiment of the present disclosure, the number of nodes of the input layer may be smaller than the number of nodes of the output layer, and the neural network may be in the form that the number of nodes decreases from the input layer to the hidden layer. Further, in the neural network according to another embodiment of the present disclosure, the number of nodes of the input layer may be larger than the number of nodes of the output layer, and the neural network may be in the form that the number of nodes increases from the input layer to the hidden layer. The neural network according to another embodiment of the present disclosure may be the neural network in the form in which the foregoing neural networks are combined.

A deep neural network (DNN) may mean the neural network including a plurality of hidden layers, in addition to an input layer and an output layer. When the DNN is used, it is possible to recognize a latent structure of data. That is, it is possible to recognize latent structures of photos, texts, videos, voice, and music (for example, what objects are in the photos, what the content and emotions of the texts are, and what the content and emotions of the voice are). The DNN may include a convolutional neural network (CNN), a recurrent neural network (RNN), an auto encoder, Generative Adversarial Networks (GAN), a Long Short-Term Memory (LSTM), a transformer, a restricted Boltzmann machine (RBM), a deep belief network (DBN), a Q network, a U network, a Siamese network, a Generative Adversarial Network (GAN), and the like. The foregoing description of the deep neural network is merely illustrative, and the present disclosure is not limited thereto.

In the embodiment of the present disclosure, the network function may include an auto encoder. The auto encoder may be one type of artificial neural network for outputting output data similar to input data. The auto encoder may include at least one hidden layer, and the odd-numbered hidden layers may be disposed between the input/output layers. The number of nodes of each layer may decrease from the number of nodes of the input layer to an intermediate layer called a bottleneck layer (encoding), and then be expanded symmetrically with the decrease from the bottleneck layer to the output layer (symmetric with the input layer). The auto encoder may perform a nonlinear dimension reduction. The number of input layers and the number of output layers may correspond to the dimensions after preprocessing of the input data. In the auto encoder structure, the number of nodes of the hidden layer included in the encoder decreases as a distance from the input layer increases. When the number of nodes of the bottleneck layer (the layer having the smallest number of nodes located between the encoder and the decoder) is too small, the sufficient amount of information may not be transmitted, so that the number of nodes of the bottleneck layer may be maintained in a specific number or more (for example, a half or more of the number of nodes of the input layer and the like).

The neural network may be trained by at least one scheme of supervised learning, unsupervised learning, semi-supervised learning, and reinforcement learning. The training of the neural network may be a process of applying knowledge for the neural network to perform a specific operation to the neural network.

The neural network may be trained in a direction of minimizing an error of an output. In the training of the neural network, training data is repeatedly input to the neural network and an error of an output of the neural network for the training data and a target is calculated, and the error of the neural network is back-propagated in a direction from an output layer to an input layer of the neural network in order to decrease the error, and a weight of each node of the neural network is updated. In the case of the supervised learning, training data labelled with a correct answer (that is, labelled training data) is used, in each training data, and in the case of the unsupervised learning, a correct answer may not be labelled to each training data. That is, for example, the training data in the supervised learning for data classification may be data, in which category is labelled to each of the training data. The labelled training data is input to the neural network and the output (category) of the neural network is compared with the label of the training data to calculate an error. For another example, in the case of the unsupervised learning related to the data classification, training data that is the input is compared with an output of the neural network, so that an error may be calculated. The calculated error is back-propagated in a reverse direction (that is, the direction from the output layer to the input layer) in the neural network, and a connection weight of each of the nodes of the layers of the neural network may be updated according to the backpropagation. A change amount of the updated connection weight of each node may be determined according to a learning rate. The calculation of the neural network for the input data and the backpropagation of the error may configure a learning epoch. The learning rate is differently applicable according to the number of times of repetition of the learning epoch of the neural network. For example, at the initial stage of the learning of the neural network, a high learning rate is used to make the neural network rapidly secure performance of a predetermined level and improve efficiency, and at the latter stage of the learning, a low learning rate is used to improve accuracy.

In the training of the neural network, the training data may be generally a subset of actual data (that is, data to be processed by using the trained neural network), and thus an error for the training data is decreased, but there may exist a learning epoch, in which an error for the actual data is increased. Overfitting is a phenomenon, in which the neural network excessively learns training data, so that an error for actual data is increased. For example, a phenomenon, in which the neural network learning a cat while seeing a yellow cat cannot recognize cats, other than a yellow cat, as cats, is a sort of overfitting. Overfitting may act as a reason of increasing an error of a machine learning algorithm. In order to prevent overfitting, various optimizing methods may be used. In order to prevent overfitting, a method of increasing training data, a regularization method, a dropout method of inactivating a part of nodes of the network during the training process, a method using a bath normalization layer, and the like may be applied.

In the meantime, according to an embodiment of the present disclosure, a computer readable medium storing a data structure is disclosed.

The data structure may refer to organization, management, and storage of data that enable efficient access and modification of data. The data structure may refer to organization of data for solving a specific problem (for example, data search, data storage, and data modification in the shortest time). The data structure may also be defined with a physical or logical relationship between the data elements designed to support a specific data processing function. A logical relationship between data elements may include a connection relationship between user defined data elements. A physical relationship between data elements may include an actual relationship between the data elements physically stored in a computer readable storage medium (for example, a permanent storage device). In particular, the data structure may include a set of data, a relationship between data, and a function or a command applicable to data. Through the effectively designed data structure, the computing device may perform a calculation while minimally using resources of the computing device. In particular, the computing device may improve efficiency of calculation, reading, insertion, deletion, comparison, exchange, and search through the effectively designed data structure.

The data structure may be divided into a linear data structure and a non-linear data structure according to the form of the data structure. The linear data structure may be the structure in which only one data is connected after one data. The linear data structure may include a list, a stack, a queue, and a deque. The list may mean a series of dataset in which order exists internally. The list may include a linked list. The linked list may have a data structure in which data is connected in a method in which each data has a pointer and is linked in a single line. In the linked list, the pointer may include information about the connection with the next or previous data. The linked list may be expressed as a single linked list, a double linked list, and a circular linked list according to the form. The stack may have a data listing structure with limited access to data. The stack may have a linear data structure that may process (for example, insert or delete) data only at one end of the data structure. The data stored in the stack may have a data structure (Last In First Out, LIFO) in which the later the data enters, the sooner the data comes out. The queue is a data listing structure with limited access to data, and may have a data structure (First In First Out, FIFO) in which the later the data is stored, the later the data comes out, unlike the stack. The deque may have a data structure that may process data at both ends of the data structure.

The non-linear data structure may be the structure in which the plurality of data is connected after one data. The non-linear data structure may include a graph data structure. The graph data structure may be defined with a vertex and an edge, and the edge may include a line connecting two different vertexes. The graph data structure may include a tree data structure. The tree data structure may be the data structure in which a path connecting two different vertexes among the plurality of vertexes included in the tree is one. That is, the tree data structure may be the data structure in which a loop is not formed in the graph data structure.

The data structure may include a neural network. Further, the data structure including the neural network may be stored in a computer readable medium. The data structure including the neural network may also include preprocessed data for processing by the neural network, data input to the neural network, a weight of the neural network, a hyper-parameter of the neural network, data obtained from the neural network, an active function associated with each node or layer of the neural network, and a loss function for training of the neural network. The data structure including the neural network may include predetermined configuration elements among the disclosed configurations. That is, the data structure including the neural network may include the entirety or a predetermined combination of pre-processed data for processing by neural network, data input to the neural network, a weight of the neural network, a hyper parameter of the neural network, data obtained from the neural network, an active function associated with each node or layer of the neural network, and a loss function for training the neural network. In addition to the foregoing configurations, the data structure including the neural network may include predetermined other information determining a characteristic of the neural network. Further, the data structure may include all type of data used or generated in a computation process of the neural network, and is not limited to the foregoing matter. The computer readable medium may include a computer readable recording medium and/or a computer readable transmission medium. The neural network may be formed of a set of interconnected calculation units which are generally referred to as “nodes”. The “nodes” may also be called “neurons.” The neural network consists of one or more nodes.

The data structure may include data input to the neural network. The data structure including the data input to the neural network may be stored in the computer readable medium. The data input to the neural network may include training data input in the training process of the neural network and/or input data input to the training completed neural network. The data input to the neural network may include data that has undergone pre-processing and/or data to be pre-processed. The pre-processing may include a data processing process for inputting data to the neural network. Accordingly, the data structure may include data to be pre-processed and data generated by the pre-processing. The foregoing data structure is merely an example, and the present disclosure is not limited thereto.

The data structure may include a weight of the neural network (in the present specification, weights and parameters may be used with the same meaning), Further, the data structure including the weight of the neural network may be stored in the computer readable medium. The neural network may include a plurality of weights. The weight is variable, and in order for the neural network to perform a desired function, the weight may be varied by a user or an algorithm. For example, when one or more input nodes are connected to one output node by links, respectively, the output node may determine a data value output from the output node based on values input to the input nodes connected to the output node and the weight set in the link corresponding to each of the input nodes. The foregoing data structure is merely an example, and the present disclosure is not limited thereto.

For a non-limited example, the weight may include a weight varied in the neural network training process and/or the weight when the training of the neural network is completed. The weight varied in the neural network training process may include a weight at a time at which a training cycle starts and/or a weight varied during a training cycle. The weight when the training of the neural network is completed may include a weight of the neural network completing the training cycle. Accordingly, the data structure including the weight of the neural network may include the data structure including the weight varied in the neural network training process and/or the weight when the training of the neural network is completed. Accordingly, it is assumed that the weight and/or a combination of the respective weights are included in the data structure including the weight of the neural network. The foregoing data structure is merely an example, and the present disclosure is not limited thereto.

The data structure including the weight of the neural network may be stored in the computer readable storage medium (for example, a memory and a hard disk) after undergoing a serialization process. The serialization may be the process of storing the data structure in the same or different computing devices and converting the data structure into a form that may be reconstructed and used later. The computing device may serialize the data structure and transceive the data through a network. The serialized data structure including the weight of the neural network may be reconstructed in the same or different computing devices through deserialization. The data structure including the weight of the neural network is not limited to the serialization. Further, the data structure including the weight of the neural network may include a data structure (for example, in the non-linear data structure, B-Tree, Trie, m-way search tree, AVL tree, and Red-Black Tree) for improving efficiency of the calculation while minimally using the resources of the computing device. The foregoing matter is merely an example, and the present disclosure is not limited thereto.

The data structure may include a hyper-parameter of the neural network. The data structure including the hyper-parameter of the neural network may be stored in the computer readable medium. The hyper-parameter may be a variable varied by a user. The hyper-parameter may include, for example, a learning rate, a cost function, the number of times of repetition of the training cycle, weight initialization (for example, setting of a range of a weight value to be weight-initialized), and the number of hidden units (for example, the number of hidden layers and the number of nodes of the hidden layer). The foregoing data structure is merely an example, and the present disclosure is not limited thereto.

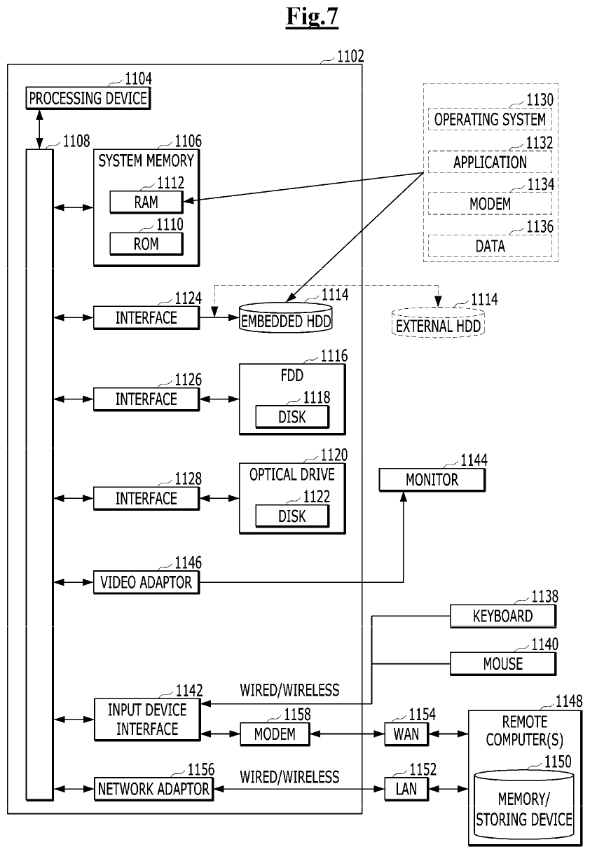

FIG. 7 is a simple and general schematic diagram illustrating an example of a computing environment in which the embodiments of the present disclosure are implementable.

The present disclosure has been described as being generally implementable by the computing device, but those skilled in the art will appreciate well that the present disclosure is combined with computer executable commands and/or other program modules executable in one or more computers and/or be implemented by a combination of hardware and software.

In general, a program module includes a routine, a program, a component, a data structure, and the like performing a specific task or implementing a specific abstract data form. Further, those skilled in the art will well appreciate that the method of the present disclosure may be carried out by a personal computer, a hand-held computing device, a microprocessor-based or programmable home appliance (each of which may be connected with one or more relevant devices and be operated), and other computer system configurations, as well as a single-processor or multiprocessor computer system, a mini computer, and a main frame computer.

The embodiments of the present disclosure may be carried out in a distribution computing environment, in which certain tasks are performed by remote processing devices connected through a communication network. In the distribution computing environment, a program module may be located in both a local memory storage device and a remote memory storage device.

The computer generally includes various computer readable media. The computer accessible medium may be any type of computer readable medium, and the computer readable medium includes volatile and non-volatile media, transitory and non-transitory media, and portable and non-portable media. As a non-limited example, the computer readable medium may include a computer readable storage medium and a computer readable transport medium. The computer readable storage medium includes volatile and non-volatile media, transitory and non-transitory media, and portable and non-portable media constructed by a predetermined method or technology, which stores information, such as a computer readable command, a data structure, a program module, or other data. The computer readable storage medium includes a RAM, a Read Only Memory (ROM), an Electrically Erasable and Programmable ROM (EEPROM), a flash memory, or other memory technologies, a Compact Disc (CD)-ROM, a Digital Video Disk (DVD), or other optical disk storage devices, a magnetic cassette, a magnetic tape, a magnetic disk storage device, or other magnetic storage device, or other predetermined media, which are accessible by a computer and are used for storing desired information, but is not limited thereto.

The computer readable transport medium generally implements a computer readable command, a data structure, a program module, or other data in a modulated data signal, such as a carrier wave or other transport mechanisms, and includes all of the information transport media. The modulated data signal means a signal, of which one or more of the characteristics are set or changed so as to encode information within the signal. As a non-limited example, the computer readable transport medium includes a wired medium, such as a wired network or a direct-wired connection, and a wireless medium, such as sound, Radio Frequency (RF), infrared rays, and other wireless media. A combination of the predetermined media among the foregoing media is also included in a range of the computer readable transport medium.

An illustrative environment 1100 including a computer 1102 and implementing several aspects of the present disclosure is illustrated, and the computer 1102 includes a processing device 1104, a system memory 1106, and a system bus 1108. The system bus 1108 connects system components including the system memory 1106 (not limited) to the processing device 1104. The processing device 1104 may be a predetermined processor among various commonly used processors. A dual processor and other multi-processor architectures may also be used as the processing device 1104.

The system bus 1108 may be a predetermined one among several types of bus structure, which may be additionally connectable to a local bus using a predetermined one among a memory bus, a peripheral device bus, and various common bus architectures. The system memory 1106 includes a ROM 1110, and a RAM 1112. A basic input/output system (BIOS) is stored in a non-volatile memory 1110, such as a ROM, an EPROM, and an EEPROM, and the BIOS includes a basic routing helping a transport of information among the constituent elements within the computer 1102 at a time, such as starting. The RAM 1112 may also include a high-rate RAM, such as a static RAM, for caching data.

The computer 1102 also includes an embedded hard disk drive (HDD) 1114 (for example, enhanced integrated drive electronics (EIDE) and serial advanced technology attachment (SATA))—the embedded HDD 1114 being configured for exterior mounted usage within a proper chassis (not illustrated)—a magnetic floppy disk drive (FDD) 1116 (for example, which is for reading data from a portable diskette 1118 or recording data in the portable diskette 1118), and an optical disk drive 1120 (for example, which is for reading a CD-ROM disk 1122, or reading data from other high-capacity optical media, such as a DVD, or recording data in the high-capacity optical media). A hard disk drive 1114, a magnetic disk drive 1116, and an optical disk drive 1120 may be connected to a system bus 1108 by a hard disk drive interface 1124, a magnetic disk drive interface 1126, and an optical drive interface 1128, respectively. An interface 1124 for implementing an outer mounted drive includes, for example, at least one of or both a universal serial bus (USB) and the Institute of Electrical and Electronics Engineers (IEEE) 1394 interface technology.

The drives and the computer readable media associated with the drives provide non-volatile storage of data, data structures, computer executable commands, and the like. In the case of the computer 1102, the drive and the medium correspond to the storage of random data in an appropriate digital form. In the description of the computer readable media, the HDD, the portable magnetic disk, and the portable optical media, such as a CD, or a DVD, are mentioned, but those skilled in the art will well appreciate that other types of computer readable media, such as a zip drive, a magnetic cassette, a flash memory card, and a cartridge, may also be used in the illustrative operation environment, and the predetermined medium may include computer executable commands for performing the methods of the present disclosure.

A plurality of program modules including an operation system 1130, one or more application programs 1132, other program modules 1134, and program data 1136 may be stored in the drive and the RAM 1112. An entirety or a part of the operation system, the application, the module, and/or data may also be cached in the RAM 1112. It will be well appreciated that the present disclosure may be implemented by several commercially usable operation systems or a combination of operation systems.

A user may input a command and information to the computer 1102 through one or more wired/wireless input devices, for example, a keyboard 1138 and a pointing device, such as a mouse 1140. Other input devices (not illustrated) may be a microphone, an IR remote controller, a joystick, a game pad, a stylus pen, a touch screen, and the like. The foregoing and other input devices are frequently connected to the processing device 1104 through an input device interface 1142 connected to the system bus 1108, but may be connected by other interfaces, such as a parallel port, an IEEE 1394 serial port, a game port, a USB port, an IR interface, and other interfaces.

A monitor 1144 or other types of display devices are also connected to the system bus 1108 through an interface, such as a video adaptor 1146. In addition to the monitor 1144, the computer generally includes other peripheral output devices (not illustrated), such as a speaker and a printer.

The computer 1102 may be operated in a networked environment by using a logical connection to one or more remote computers, such as remote computer(s) 1148, through wired and/or wireless communication. The remote computer(s) 1148 may be a work station, a computing device computer, a router, a personal computer, a portable computer, a microprocessor-based entertainment device, a peer device, and other general network nodes, and generally includes some or an entirety of the constituent elements described for the computer 1102, but only a memory storage device 1150 is illustrated for simplicity. The illustrated logical connection includes a wired/wireless connection to a local area network (LAN) 1152 and/or a larger network, for example, a wide area network (WAN) 1154. The LAN and WAN networking environments are general in an office and a company, and make an enterprise-wide computer network, such as an Intranet, easy, and all of the LAN and WAN networking environments may be connected to a worldwide computer network, for example, the Internet.

When the computer 1102 is used in the LAN networking environment, the computer 1102 is connected to the local network 1152 through a wired and/or wireless communication network interface or an adaptor 1156. The adaptor 1156 may make wired or wireless communication to the LAN 1152 easy, and the LAN 1152 also includes a wireless access point installed therein for the communication with the wireless adaptor 1156. When the computer 1102 is used in the WAN networking environment, the computer 1102 may include a modem 1158, is connected to a communication computing device on a WAN 1154, or includes other means setting communication through the WAN 1154 via the Internet. The modem 1158, which may be an embedded or outer-mounted and wired or wireless device, is connected to the system bus 1108 through a serial port interface 1142. In the networked environment, the program modules described for the computer 1102 or some of the program modules may be stored in a remote memory/storage device 1150. The illustrated network connection is illustrative, and those skilled in the art will appreciate well that other means setting a communication link between the computers may be used.

The computer 1102 performs an operation of communicating with a predetermined wireless device or entity, for example, a printer, a scanner, a desktop and/or portable computer, a portable data assistant (PDA), a communication satellite, predetermined equipment or place related to a wirelessly detectable tag, and a telephone, which is disposed by wireless communication and is operated. The operation includes a wireless fidelity (Wi-Fi) and Bluetooth wireless technology at least. Accordingly, the communication may have a pre-defined structure, such as a network in the related art, or may be simply ad hoc communication between at least two devices.

The Wi-Fi enables a connection to the Internet and the like even without a wire. The Wi-Fi is a wireless technology, such as a cellular phone, which enables the device, for example, the computer, to transmit and receive data indoors and outdoors, that is, in any place within a communication range of a base station. A Wi-Fi network uses a wireless technology, which is called IEEE 802.11 (a, b, g, etc.) for providing a safe, reliable, and high-rate wireless connection. The Wi-Fi may be used for connecting the computer to the computer, the Internet, and the wired network (IEEE 802.3 or Ethernet is used). The Wi-Fi network may be operated at, for example, a data rate of 11 Mbps (802.11a) or 54 Mbps (802.11b) in an unauthorized 2.4 and 5 GHz wireless band, or may be operated in a product including both bands (dual bands).

Those skilled in the art may appreciate that information and signals may be expressed by using predetermined various different technologies and techniques. For example, data, indications, commands, information, signals, bits, symbols, and chips referable in the foregoing description may be expressed with voltages, currents, electromagnetic waves, magnetic fields or particles, optical fields or particles, or a predetermined combination thereof.

Those skilled in the art will appreciate that the various illustrative logical blocks, modules, processors, means, circuits, and algorithm operations described in relationship to the embodiments disclosed herein may be implemented by electronic hardware (for convenience, called “software” herein), various forms of program or design code, or a combination thereof. In order to clearly describe compatibility of the hardware and the software, various illustrative components, blocks, modules, circuits, and operations are generally illustrated above in relation to the functions of the hardware and the software. Whether the function is implemented as hardware or software depends on design limits given to a specific application or an entire system. Those skilled in the art may perform the function described by various schemes for each specific application, but it shall not be construed that the determinations of the performance depart from the scope of the present disclosure.

Various embodiments presented herein may be implemented by a method, a device, or a manufactured article using a standard programming and/or engineering technology. A term “manufactured article” includes a computer program, a carrier, or a medium accessible from a predetermined computer-readable storage device. For example, the computer-readable storage medium includes a magnetic storage device (for example, a hard disk, a floppy disk, and a magnetic strip), an optical disk (for example, a CD and a DVD), a smart card, and a flash memory device (for example, an EEPROM, a card, a stick, and a key drive), but is not limited thereto. Further, various storage media presented herein include one or more devices and/or other machine-readable media for storing information.

It shall be understood that a specific order or a hierarchical structure of the operations included in the presented processes is an example of illustrative accesses. It shall be understood that a specific order or a hierarchical structure of the operations included in the processes may be rearranged within the scope of the present disclosure based on design priorities. The accompanying method claims provide various operations of elements in a sample order, but it does not mean that the claims are limited to the presented specific order or hierarchical structure.

The description of the presented embodiments is provided so as for those skilled in the art to use or carry out the present disclosure. Various modifications of the embodiments may be apparent to those skilled in the art, and general principles defined herein may be applied to other embodiments without departing from the scope of the present disclosure. Accordingly, the present disclosure is not limited to the embodiments suggested herein, and shall be interpreted within the broadest meaning range consistent to the principles and new characteristics presented herein.

What is claimed is:

1. A method of augmenting data associated with a target protein, the method performed by a computing device, the method comprising:
obtaining a target protein and indicator information related to the target protein included in training data;
identifying a homologous protein of the target protein; and
augmenting the training data by matching the homologous protein to the indicator information related to the target protein.

2. The method of claim 1, wherein the indicator information related to the target protein includes affinity information between the target protein and a drug.

3. The method of claim 2, further comprising:
filtering the augmented training data by considering the affinity information between the target protein and the drug and the affinity information between the homologous protein and the drug.

4. The method of claim 3, wherein the filtering includes comparing affinity information between the target protein and the drug given from training data or predicted by a deep learning model, and affinity information between the homologous protein and the drug predicted by the deep learning model in a current batch of training data.

5. The method of claim 4, wherein the filtering includes filtering data regarding the homologous protein having accuracy of a specific ranking or more among accuracy values which the homologous proteins in the batch have, and
the accuracy values are generated based on a comparison between the affinity information between the homologous protein and the drug predicted by the deep learning model, and the affinity information between the target protein and the drug given from training data or predicted by the deep learning model.

6. The method of claim 3, wherein the filtering includes performing filtering for the augmented training data from a middle of a training process of a deep learning model which is currently trained.

7. The method of claim 1, wherein the identifying of the homologous protein of the target protein further includes performing multiple sequence alignment (MSA) for the target protein and multiple homologous proteins.

8. The method of claim 7, wherein the performing of the MSA includes performing a search for the target protein and multiple homologous proteins satisfying a predetermined matching degree ratio.

9. A computer program stored in a non-transitory computer-readable storage medium, wherein the computer program executes the following operations for augmenting data associated with a target protein when the computer program is executed by one or more processors, the operations comprising:
an operation of obtaining a target protein included in training data and indicator information related to the target protein;
an operation of identifying a homologous protein of the target protein; and
an operation of augmenting the training data by matching the homologous protein to the indicator information related to the target protein.

10. A computing device for augmenting data associated with a target protein, comprising:
at least one processor; and
a memory,
wherein at least one processor is configured to
obtain a target protein included in training data and indicator information related to the target protein,
identify a homologous protein of the target protein, and
augment the training data by matching the homologous protein to the indicator information related to the target protein.
